(19) United States
(12) Patent Application Publication
(10) Pub. No.: US 2011/0145982 A1
(43) Pub. Date: Jun. 23, 2011

(54) EYE PROTECTION FOR ATHLETIC USES

(76) Inventors: Zachary B. Fine, Charlotte, NC (US); Daniel L. Bizzell, Charlotte, NC (US)

(21) Appl. No.: 12/841,958

(22) Filed: Jul. 22, 2010

Related U.S. Application Data
(60) Provisional application No. 61/227,762, filed on Jul. 22, 2009.

(51) Int. Cl. A61F 9/02

(52) U.S. Cl. 2/431

(57) ABSTRACT

A protective eyewear system suitable for use during athletic competitions includes a hybrid frame that features an elastomerically flexible portion and rigid portions and a pair of lenses independently supported within the rigid portions. The lenses are generally concave to the eyes, but the rigid portions that support and surround the lenses are generally convex to the eyes. The rigid portions also include armatures that confine interaction between the rigid portions and the eye sockets to certain specific areas that are more likely to be able to withstand impact. The flexible portion provides padding and comfort. Various ventilation measures prevent fogging of the lenses and sweat retention.

### CROSS-REFERENCE TO RELATED APPLICATIONS

This application claims the benefit of U.S. Provisional Patent Application Ser. No. 61/227,762, filed Jul. 22, 2009, the entirety of which is incorporated herein by reference.

### FIELD OF THE INVENTION

The present invention relates generally to protective eyewear for use in athletic competitions, principally soccer (known internationally as football), and more particularly to athletic eyewear that features protected independent lenses, facial contouring, elastomeric flexing and shock attenuation, an eye-protective lens design, and adjustability to a wide range of sizes.

### BACKGROUND OF THE INVENTION

Eye protection is an increasingly important aspect of athletic competition. Maintaining good eyesight is usually essential to maintaining athletic competitiveness, whether at the highest professional levels, at the most casual amateur levels, or at any level in between. Awareness of the dangers posed to eyes during competition, particularly among youth participants, has led to a desire on the part of event and program organizers, parents, and participants for eye protection that aids performance, is comfortable during competition, and that does not cause damage of a more serious nature.

Various apparatuses for protecting the eyes during athletic competitions are well known. In sports such as baseball and golf, in which contact with the eyes is rare, ordinary eyeglasses and sunglasses are allowed to be used and are frequently in use, whether for vision correction or sun protection. In more contact-oriented sports such as football and basketball, ordinary eyewear is more easily broken and can cause more serious injury than if no eyewear was worn. Thus, more specialized eye protection schemes for those sports are available, including, for example, shields embedded in a football helmet's face mask, face shields, or glasses/goggles hybrids that are designed to withstand moderate impact.

In sports such as cycling and athletics (track and field), in which contact is not a part of the competition but accidentally occurs, the use of ordinary eyewear can pose the same risk as that posed by its use in contact sports, but specialized eyewear either is structurally impracticable or interferes too greatly with ordinary competition to be used. For example, a cyclist who wears ordinary polycarbonate-lens sunglasses during a race could easily lose an eye or suffer a grave and deep cut if, during a crash, the edge of the lens slices through the skin around the eye or into the eye itself. By contrast, bulkier specialized eyewear may interfere with the aerodynamics of cycling, causing the cyclist a slight disadvantage that, at the highest levels of competition, can mean the difference between winning and losing.

Conventional athletic eyewear suffers from a number of deficiencies that can render it ineffective at achieving the protection desired by participants. For example, flexibility of the eyewear apparatus is desirable to allow closer contouring of the eyewear to the user's face, thus eliminating blind spots and improving aerodynamic response. However, conventional methods of providing flexibility have largely required the use of two or three disparate principal materials: a soft rubber, silicone, or elastomeric superstructure; a polycarbonate or other plastic lens; and, in some cases, a metal substructure or frame to support the lenses or provide stability for the eyewear. Under those circumstances, the tendency during contact is for the polycarbonate lens and/or the metal substructure to slice through the superstructure and to cut the eye, eyelid, brow, or cheek. Even without impact, consistently rough wear leads to premature breakdown.

In a sport such as soccer (football), the player's head is frequently used to strike the ball. The use of eyewear by soccer players can interfere with play by producing a different ball trajectory than intended. Alternatively, impact with the ball can dislodge conventional eyewear. For those reasons, soccer players tend toward a strong preference not to wear eyewear due to the performance issues. More seriously, heading can result in repeated impact by the ball (or with other players) that damages the eyewear and exposes the player to the very kind of injury he or she was trying to prevent. Consequently, the governing bodies for soccer have largely refused to permit players to wear eyewear during competitions, allowing only a few players at most to wear eyewear only with the gravest of justifications, such as, for example, a medical condition that poses a serious risk if the eyes are subjected to impact.

What is needed, then, is an eye protection device that is suitable for use in many types of athletic events (whether or not impact with the player's head is part of the game) that features protected lenses to prevent injury, that contours to the face to prevent interference with game play and allows improved peripheral vision, that provides flexing and shock attenuation, and that adjusts to a wide range of sizes to promote wide adoption.

### SUMMARY OF THE INVENTION

In accordance with the aforementioned needs and other needs, the present invention includes an item of protective eyewear suitable for use during athletic competitions. The eyewear generally includes a pair of lenses independently supported within a hybrid frame, the hybrid frame including both rigid and elastomerically flexible portions. The lenses are generally concave to the eye, but they are each set within a rigid frame portion or exoskeleton of varying width.

The exoskeleton is configured such that its wider points, denoted as armatures or aprons, correspond to and interface with portions of the eye socket at which pressure may be safely applied in the event of impact, namely, at the inner brow, the outer corner, and the cheekbone. The rigid frame portion narrows at the other points in order to support the lens without causing additional points of pressure against the orbit during impact. The rigid frame portion is further configured to be convex to the eye and contoured to the face, such that in an impact upon the eyewear, the applied force is directed outwardly, rather than the conventional concave-to-eye arrangement that causes force to be directed toward the eye socket.

The elastomerically flexible portion of the hybrid frame is configured to connect the two lenses in a manner that permits them to flex and float independently, as well as interposing a portion of the elastomeric material between the rigid portion of the hybrid frame and the eye socket and the bridge of the nose, so as to provide shock attenuation, protection, and greater comfort.

The combination of the rigid and flexible portions of the hybrid frame allows for a reasonably stiff, yet resilient frame.

In a further feature of the eyewear, the lenses may include sun protection, including protection against UV rays as well as tinting for comfort or increased utility. For example, the lenses may be tinted yellow or blue to achieve certain desired optical effects and enhancements, or they may be tinted virtually any color for aesthetic reasons.

In another feature of the eyewear, the hybrid frame may include side extension portions that feature a novel connection system, in which the ends of a support strap may be inserted through a selected one of a series of apertures, in order to vary the circumferential size of the eyewear to match it to the size of the wearer's head. These apertures may be disposed directly in the elastomer that forms the side extension portions, or they may be disposed in a hardgoods reinforcing member that makes the side extension more durable at the point of interface with the strap.

In still another feature of the eyewear, the hybrid frame is highly contoured to the wearer's head and face and is designed for close wear, the hybrid frame being relatively thin and thus unlikely to interfere, for example, with a soccer player's ability predictably to head the ball in the same manner as a player who is not wearing the eyewear of the invention. This arrangement also allows for improved peripheral vision than is possible with conventional eyewear.

The present invention also includes custom eyewear in which the components described above are custom-fit to a particular user, along with a method of forming that custom eyewear that includes the steps of taking measurements of the user's eye orbits and placing and shaping the armatures and, optionally, any other points of contact to provide a maximally customized fit.

The present invention further includes a lens configuration that includes one or more cutouts or voids in the lens area, which voids permit additional ventilation and help to prevent fogging of the lenses. To promote additional ventilation and to provide additional shock absorption, the interior portion of the softgoods interfacing with the user's head may be substantially thickened and provided with a number of channels that permit ventilation and evacuation of sweat.

In another feature of the invention, the side extension members may include additional contouring to ensure a better, more secure fit around the ear. This feature also accommodates an alternative angling of the side extension members that permits the lens tilt to be varied to particular purposes, such as optimized air flow or increasing the distance between the lens and the face.

### BRIEF DESCRIPTION OF THE DRAWINGS

Further features, embodiments, and advantages of the present invention will become apparent from the following detailed description with reference to the drawings, wherein:

FIG. 1 is an environmental view of a preferred embodiment of the present invention;

FIG. 2 is a general perspective view of a device as in FIG. 1;

FIG. 3 is an exploded view of a device as in FIG. 2, to show the arrangement of the components;

FIG. 4 is a detail view depicting an adjustable strap feature of the present invention;

FIG. 5 is a lateral detail view of a preferred embodiment of the present invention;

FIGS. 6A-6B are sequential views of the device as above, under impact;

FIG. 7 is a detail view depicting a concave-convex feature of the present invention;

FIG. 8 is an isometric comparison of the device as above, before and after flexing;

FIGS. 9A-9B are isometric views of side impact of a soccer ball upon the device as above during directional heading;

FIG. 10 is a detail view depicting an alternative lens configuration;

FIG. 11 is a detail view depicting an alternative ventilation feature of the present invention; and

FIG. 12 is a detail view depicting an alternative configuration of the earpiece.

### DETAILED DESCRIPTION OF THE PREFERRED EMBODIMENTS

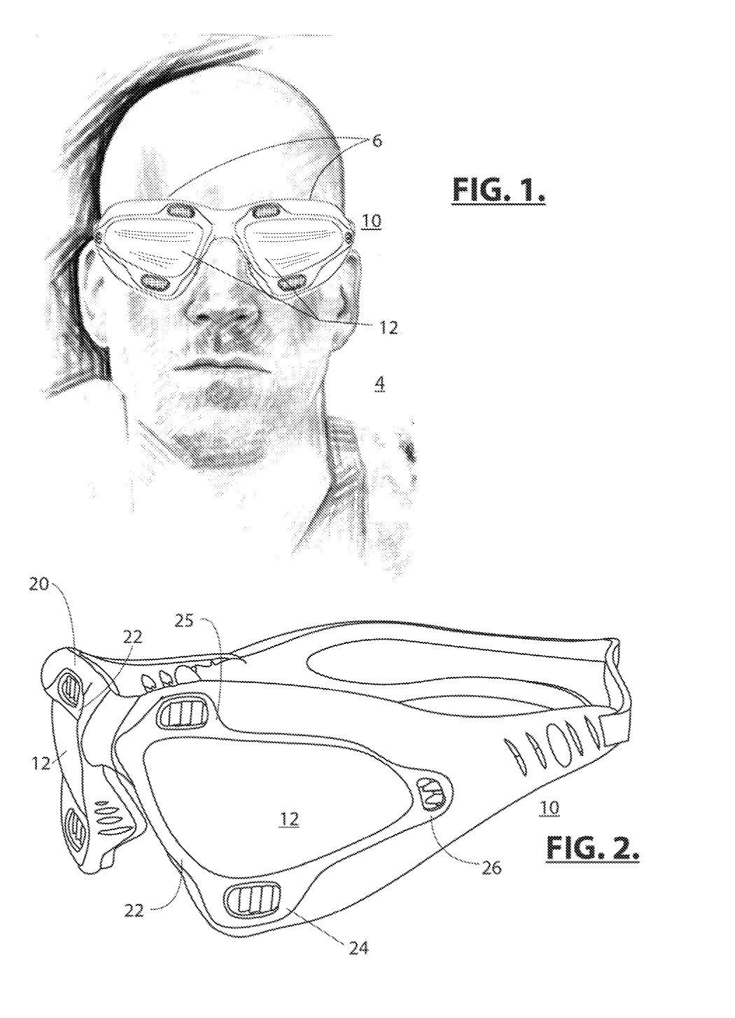

Referring now to the drawings, and particularly to FIG. 1, the present invention is depicted in an environmental view in use by a wearer 4. In a preferred embodiment a set of eyewear 10 may also be conveniently referred to as “sunglasses,” “glasses,” or “optics.” Two lenses 12 are preferably formed of polycarbonate or another suitable material and are generally concave to the eye (not shown) of the wearer. The lenses 12 may be tinted or coated, may be provided with a UV-filtering material, and may be polarized to reduce glare, all in manners that are well known in the art to which the present invention relates.

Referring now also to FIG. 2, the lenses 12 are each disposed within a hybrid frame 20. The frame 20 comprises both soft and hard parts, to be described in greater detail below, which parts serve the novel purposes of the present invention in various ways. The hard or rigid portions 22 of the frame 20 surround and support each of the lenses 12, providing a mechanism for containment of the rigid plastic lenses 12, generally by compression or interference fit, although other mechanisms such as welding or gluing may be employed without departing from the scope of the invention. It should be noted that the rigid portions are not rigid in the sense that they are completely inflexible, but rather that they are made of a comparatively hard plastic and are less flexible than the flexible portions. The combination of the rigid and soft portions of the frame serves to provide structure to the optics while accomplishing the objectives of the invention.

The rigid portions 22 retain the lenses 12 and alter the nature of the interface between the lenses 12 and the eye socket 6 in several specific ways. First, the rigid portions 22 vary in width as they traverse the circumference of the lenses 12. At some points, the rigid portion 22 is thin, in order to provide at least the minimum support of the lens structure necessary for safety purposes. At other points, the rigid portion 22 widens into aprons or armatures 24,25,26 extending laterally outward from the edge of the lens 12. The arrangement of these armatures 24,25,26 is such that the effect is to present a convex interface to the eye socket 6. Thus, during impact upon the eyewear 10 by, for example, a soccer ball, the tendency is for the force of the ball to be deflected outward; as the lens 12 flexes in, the point of interface with the eye socket 6 (the rigid portion 22) flexes out, directing the force outward instead of into the eye socket 6.

This arrangement thus differs from conventional glasses partly in that in the present invention the eye-optics interface is convex to the eye and orbit, while conventional glasses are concave thereto. It also differs from conventional goggles partly in that in the present invention the eye-optics interface is disposed about the orbit, while conventional goggles generally rest interior of the orbit.

Second, the armatures 24,25,26 are positioned to interface with the eye socket at locations where the underlying bone structure is best able to withstand an impact, namely at the inner brow, the outer corner, and the cheekbone, respectively. By spreading the force of an impact over a larger surface area than conventional arrangements, and by positioning that impact upon portions of the eye socket 6 that can withstand the impact, the likelihood of cutting, bone breakage, bruising, and other ills associated with conventional eyewear is substantially reduced.

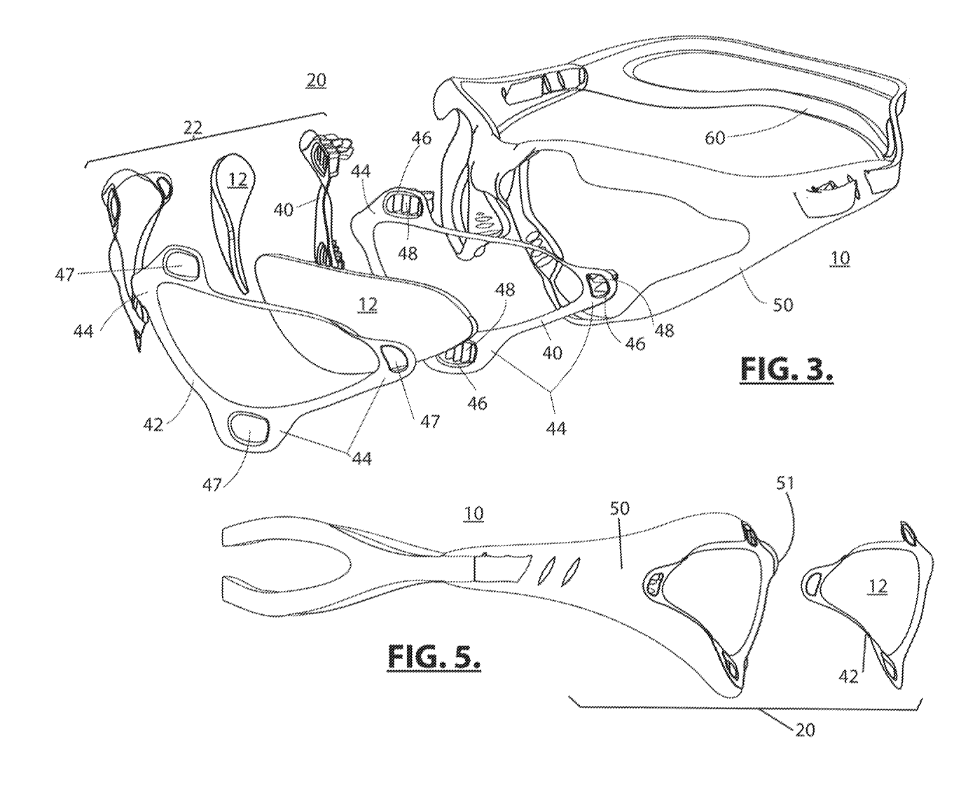

The rigid portions 22 may be described as exoskeletal in nature, in that together with the soft portions 50 (see FIG. 3) they provide external support and protection for the eye socket and bridge of the nose during impact.

Referring now to FIG. 3, the eyewear 10 depicted in previous illustrations is shown with the components thereof in exploded relation. The lens 12 can now be seen as cooperating with the rigid-plastic frame portion 22, and is more specifically placed between a support member 40 and a bezel 42. The support member 40 and bezel 42 cooperate to support and hold the lens 12 in place, and may be provided with edge-based recesses or other means by which the lens 12 may be fit snugly between the support member 40 and bezel 42, whether by interference, compression, or another method. The support member 40 and bezel 42 are preferably designed to snap together about the lens 12 to form a single lens unit.

In one embodiment of the present invention, the snap-in arrangement of the bezel 42 and lens 12 can be used to facilitate the changing of the lenses 12. In this manner, the same frame could be used to hold interchangeable lenses, such as lenses of different tinting strengths or colors, precision-ground prescription optics, and the like. Alternatively, if prescription optics are needed, special provision in the construction of the frame 20 could be made to allow prescription lenses to be added between the polycarbonate lenses 12 and the eye, although the optics of the present invention are particularly suited to use in conjunction with contact lenses.

As can be seen in the drawing, both the support member 40 and bezel 42 have aprons or armatures 44 (corresponding to elements 24,25,26 in FIG. 2). In the preferred embodiment of the present invention depicted in FIG. 3, the armatures 44 are provided with ventilation apertures 46,47. These ventilation apertures 46,47 are placed to allow air flow to the eye socket area, thereby reducing the chances of the lenses becoming fogged due to perspiration. The apertures 46 on the support member 40 may be provided with fins 48 for added stability and rigidity.

Preferably at least one of the ventilation apertures 46,47 is disposed at the lowest point of the frame in order to permit liquids such as perspiration to drain from behind the lenses.

The placement and shaping of the armatures 44 will be reasonably standardized in a preferred embodiment. However, it is equally possible and within the scope of the present invention to provide customized eyewear. In such eyewear, the various components described above, and particularly including the shape and placement of the armatures 44 and the soft sections 50 of the frame 20 will be custom-fit to a particular user. In order to form the eyewear, the steps of a method according to the present invention will involve taking measurements of the user's eye orbits and other points of interface with the eyewear and placing and shaping the armatures and any other points of contact to provide a maximally customized fit.

Also shown in FIG. 3 is a more detailed depiction of the so-called “soft” section 50 of the frame 20. The soft section 50 is preferably composed of an elastomeric foam of sufficient strength and stability to provide support for the lenses 12 and the rigid frame portion 22, but soft enough to provide resilient flexing during use, as well as a degree of shock attenuation during impact. This latter purpose remains significant despite the manner in which shock forces are distributed about the orbit of the eye due to the novel armature design 24,25,26 (see FIG. 2). The elastomeric soft section 50 provides an additional degree of cushioning about the whole eye socket that further protects the user during impact, reduce the flexing force applied to the hardgoods 22, and provide considerably greater comfort in general use, particularly in view of the tendency, perhaps necessity, of wearing the eyewear 10 tightly against the eyes for security or reliability purposes.

A further advantage of the soft section 50 is that its nature permits the eyewear 10 to conform more closely to the contour of the user's head, forming a sort of wraparound arrangement. Because the soft portion 50 of the frame 20 is flexible, it further permits the lenses 12 to move independently of each other, an arrangement that presents a notable advantage over prior-art designs that rely upon a single rigid frame for containing the lenses. Because the lenses 12 may move independently, an impact on one side versus another, such as when a soccer player is heading the ball from one side of the head, is unlikely to dislodge the opposite lens 12 from its proper location. This arrangement allows for improved performance of the eyewear during heading. Moreover, the lenses' capacity for independent movement, together with other features of the invention, allows for a tight, snug fit, which is of particular importance in soccer to enable directional heading.

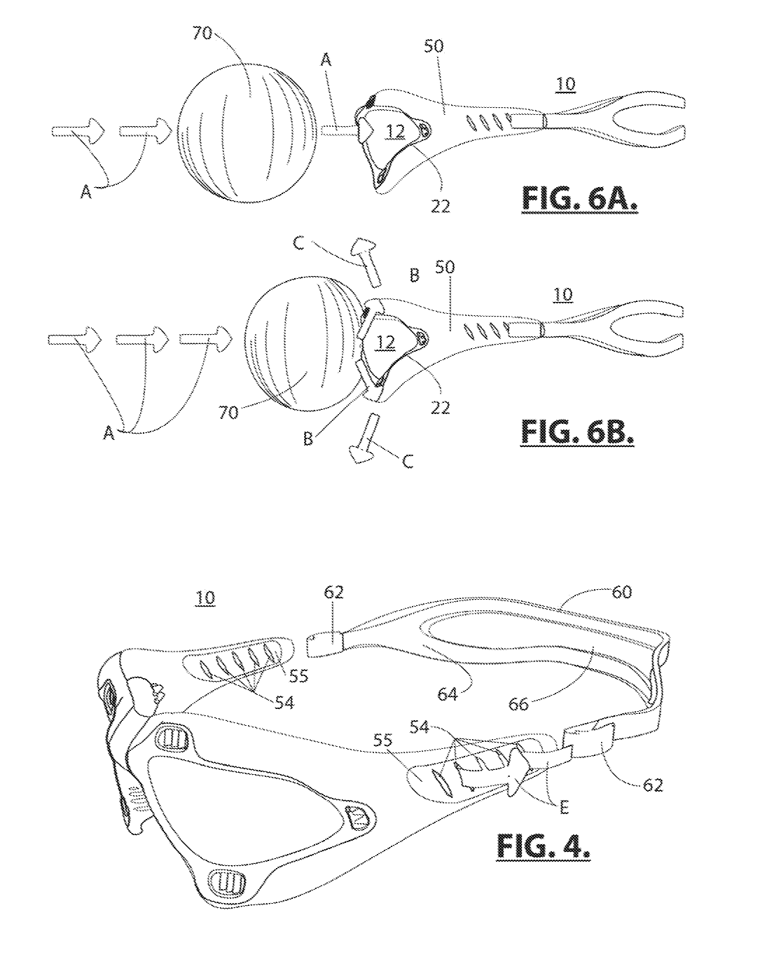

As can be seen in FIG. 3, an adjustable strap 60 further enables close, custom placement of the lenses against the eye socket. The adjustable strap feature is shown in greater detail in FIG. 4.

In FIG. 4, the adjustable strap 60 is composed of a resilient elastomer of an appropriate length sufficient to complete the circumference of the user's head. The strap 60 includes two ends 62 with a main body portion 64 therebetween. In the preferred embodiment depicted in the figure, the main body portion may include a vent 66 that runs substantially all of its length. The vent 66 serves to improve comfort, enables a player with long hair to place a ponytail therethrough, and allows for a more secure and stable fit through placement of the two strap segments, thus reducing the need for multiple sizes.

The ends 62 are designed to cooperate with a series of slits 54 present in the side members or ear pieces 52 of the elastomer 50. The slits 54 are disposed sequentially along the side members 52, such that by choosing the slit 54 through which the end 62 of the strap 60 is placed, the circumference of the eyewear 10 can be varied. To secure the strap 60, the end 62 is inserted in the direction of arrow E through one of the slits 54, then back through another of the slits 54, then optionally through yet another of the slits 54 in order to secure the strap end 62. This arrangement provides a secure fit for the strap 60 by preventing it from being easily dislodged simply through resilience of the materials. This arrangement also allows the strap 60 to be tightened to whatever degree is necessary, regardless of the slits 54 chosen. In that situation, the additional slits 54 may serve as further ventilation points.

Rather than disposing the slits 54 directly in the softgoods 50, it may be preferable to provide a reinforcing member 55, embedded within the softgoods 50, in which the slits 54 are disposed. This arrangement provides for a more durable product that is resistant to tearing, particularly when the optics are donned or removed, by preventing the weakening of the elastomer 50 at the point of interface with the strap 60. Those skilled in the art will recognize that the resilient stretching of the elastomer 50 and the strap 60 will allow the eyewear 10 to be placed over the user's head while allowing for facial contouring, an ergonomic arrangement, and, if required or desired, a tight, snug fit during athletic play.

Referring now to FIG. 5, a lateral view of eyewear 10 according to the present invention is illustrated, with like numerals corresponding to those features of the invention already discussed. As can be seen in this lateral view, in which the lens 12 and bezel 42 are exploded from the frame 20, the eyewear 10 is arranged as a wraparound design in order to allow contouring to the usual shape of a user's head. However, it should be noted that the resilient flexing of the elastomer 50, particularly at the point 51 that connects the two lenses 12, allows for a variable angle of attack with respect to the user's head. This arrangement allows for a great deal of customization to head shape, in a manner that would not be possible with a one-piece hardgoods frame 22.

FIGS. 6A and 6B are sequential views of a device according to the present invention, in which the impact of a ball 70 upon the eyewear 10 is shown. The ball 70 as shown in the figure is the size of a tennis ball or racquet ball, although the principles described herein will apply to other ball sizes, up to and including soccer balls and basketballs, as well as to more rigid balls such as baseballs. In FIG. 6A, the ball 70 is approaching the eyewear 10 with significant velocity in the direction of the arrows A. In a conventional approach to eyewear, the impact depicted in FIG. 6B could be devastating, potentially resulting in a shattered lens, a serious facial cut (whether from a lens edge or the frame), significant orbital bruising or breakage, or damage to the eye. Even if there were no serious damage, significant pain could result. In any event, the user might experience a full range of harms, from minor pain that distracts the user from the competition, to temporary but manageable injury, to permanent disability or, in extreme cases, even death.

The present invention, however, mitigates this kind of impact in a number of ways. First among them is the presence of significantly softer materials 50 about the eye and nose. These soft sections 50 provide for the absorption of some of the force of the ball 70. Second, the armatures 44 provide wide areas of support for the eyewear against those portions of the orbital bone of the user that are most able to withstand the force. This arrangement directs most of the force to be applied in those areas and spread over a wider area than, for example, the edge of the lens or a narrow frame (as in some conventional arrangements). Finally, although the lenses 12 are themselves concave to the eye, the hardgoods frame portion 22 surrounding the lens 12 is actually convex to the eye. Upon impact of the ball 70 against the eyewear 10, any force applied to the concave lens 12 is directed in the direction of arrows B to the convex hardgoods frame portion 22. Because of the convex arrangement of the hardgoods frame portion 22, a substantial portion of the impact force is directed outward, away from the eye, in the direction of arrows C.

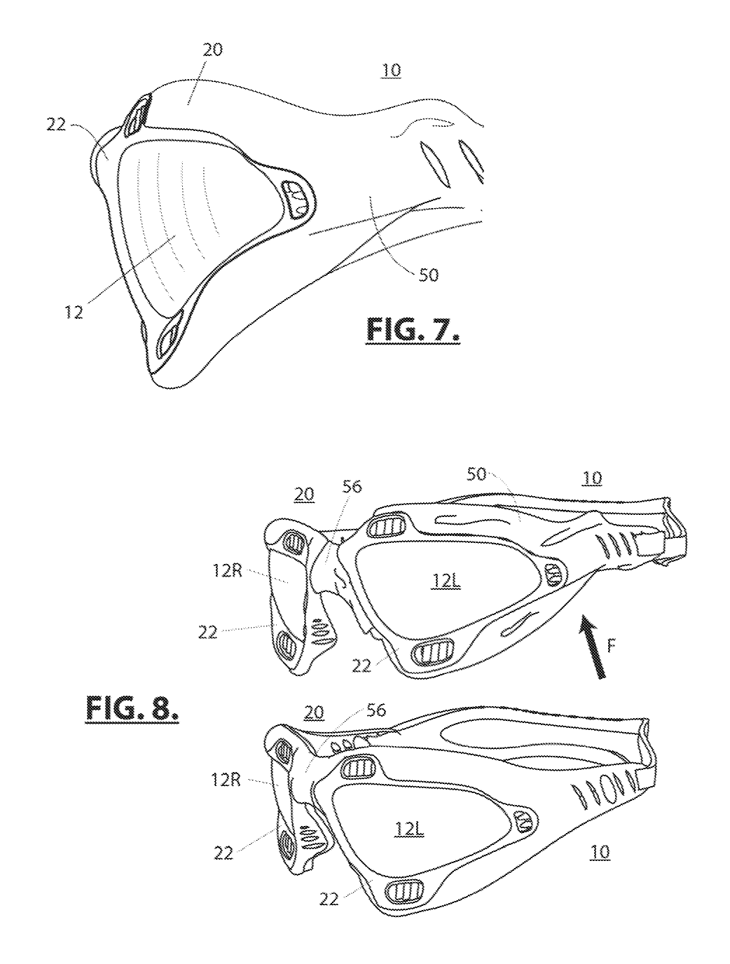

Referring now to FIG. 7, the precise arrangement of a preferred embodiment of the present invention, and more particularly of the concave-convex arrangement of the eyewear 10, is shown in a detail view. The lens 12 is arranged conventionally as concave to the eye, an arrangement that is necessary, among other reasons, to prevent distortion through the lens. However, the hardgoods portion 22 of the frame 20, though designed in a preferred embodiment for a precise fit to surround and protect the lens edge, is itself convex to the face or eye. This disposition of the lens 12 and frame portion 22 accommodates the shock attenuation efforts previously described by providing for the direction of forces during impact in specific ways as described.

Referring now to FIG. 8, eyewear 10 is shown isometrically in a sequential view to illustrate flexing of the soft sections 50, which promotes independent movement of the hardgoods portion 22 of the frame 20. As a force is applied to only one side of the eyewear 10 in the direction of arrow F, the elastomeric composition of the soft sections 50 permits movement of the left lens 12L in response to this force, largely without disturbing the placement and orientation of the right lens 12R. The advantages of this arrangement, in addition to those described above, are that under normal wear the lenses 12L,12R may be fit to the precise contours of the wearer's face, and that under (for example) a side impact such as one that would apply a force in the direction of arrow F, the lenses 12L,12R move independently, meaning that the right lens 12R stays in place even if the left lens 12L is somewhat dislodged.

In order to facilitate the independent flexing function, those skilled in the art will recognize that the center portion 56 of the frame 20, typically referred to as the “nose piece” because it is designed to fit over and around the bridge of the user's nose, may be entirely or primarily constituted as elastomer, and more particularly and preferably the same elastomeric flexible material as the remainder of the soft portion 50.

The soft portion 50 of the frame 20 may be conveniently formed through molding. In a preferred embodiment, the hardgoods portion 22 will be placed into a mold and the elastomeric foam comprising the soft portion 50 will be injected into the form, around the hardgoods 22 in a predetermined manner, to form a unified piece. The elastomer portion 50 may be textured or perforated in order to assist in the management of perspiration, for example.

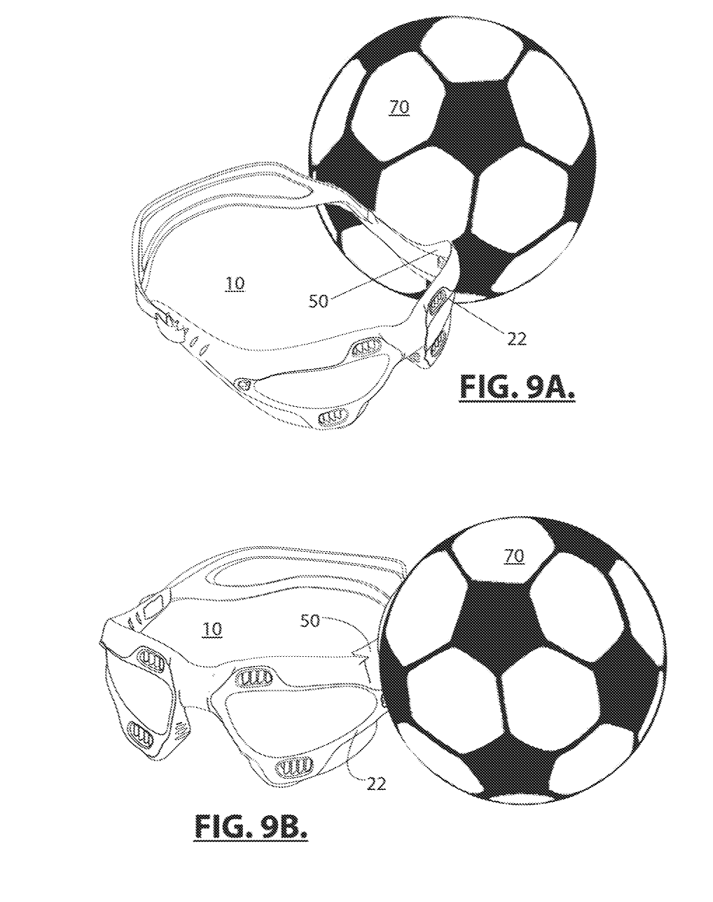

Referring now to FIGS. 9A-9B, the impact of a soccer ball 70 upon the optics 10 during directional heading is illustrated in opposite-angle views. During impact, the elastomeric portion 50 of the frame tends to collapse flat (but resiliently so) as the force of impact is attenuated. Because at the point of impact the elastomeric portion 50 is essentially flat, the arrangement is as close to a “natural” (optics-free) heading position as can be obtained with optics in place. The result is that the player's ability to control the direction of rebound and, conversely, to avoid an undesired rebound direction, is substantially improved.

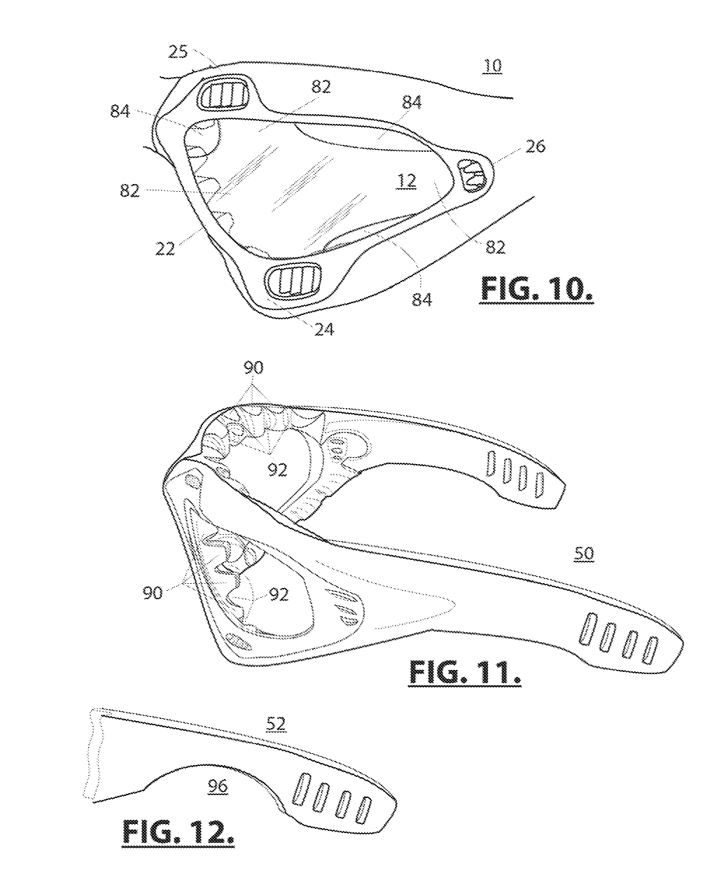

Referring now to FIG. 10, an alternative configuration for the lens 12 is shown. In other embodiments of the present invention, the lens 12 extends into full, uninterrupted contact with the rigid portion 22 of the frame 20. In the embodiment shown in FIG. 10, the lens 12 is attached to the rigid portion 22 of the frame 20 only at selected points 82, such that additional apertures 84 are provided. One purpose of these apertures 84 is to provide additional means for ventilating the region between the optics 10 and the user's face (i.e., behind the lens), in order to prevent fogging due to excessive moisture in that region. The particular placement of these apertures 84 can be varied according to a particular design need, although it is preferable to have the points of attachment 82 between the lens 12 and the rigid portion 22 in the area of the armatures 24,25,26 to ensure safe operation and optimal ventilation of the optics 10. The combination of the apertures 84 with the nodules 90 (see FIG. 11) and selection of an appropriate lens tilt provides optimal operation, ventilation, and moisture management during athletic competitions.

Referring now to FIG. 11, an alternative embodiment of the softgoods portion 50 of the frame 20 is shown. In this embodiment, the softgoods portion 50 contains a number of nodules 90 that project between the optics and the user, so as to provide additional cushioning against, for example, the blow from a soccer ball. This additional cushioning is constituted as the nodules 90 because doing so creates a series of channels 92 through which perspiration may flow and be directed away from the user's eye region. These channels 92 also aid in ventilation, helping to prevent fogging that can interfere with the user's vision while wearing the optics during a sporting event. This channeling makes for more efficient removal of sweat and humid air, particularly throughout the nose area.

Referring now to FIG. 12, an alternative embodiment of the side member 52 is illustrated, in which additional contouring is present. Specifically, the side member is provided with an ear cutout 96, which may be necessary when considering the particular disposition of the side members 52 in relation to the lens tilt and the overall operation of the optics 10. Allowing for an ear cutout 96 promotes both comfort and proper placement of the optics during wear, particularly in applications where it may be helpful to move the preferred placement of the lens 12 farther from the user's face.

Overall, the present invention represents a novel approach to several continuing problems with conventional optics. The concave-convex design of the eyewear, the elastomeric flexing of the soft portion, and the strategically placed armatures serve to provide significant shock attenuation, with attendant safety and comfort advantages, particularly in sports and activities in which impacts upon the head or eyes are possible, if not desirable. The eyewear also provides significant advantages over prior art designs in terms of adjustability and contouring to the user's face, in a manner that allows for a custom, ergonomic, anthropometric fit even among users of varying head sizes and shapes. Moreover, the presence in the eyewear of two independent, yet fully protected lenses operates to ensure that impacts upon one of the lenses will not adversely affect the other lens, thereby maintaining the safety advantages of the lenses during impact. Other features of the invention take account of the use of these optics in athletic competitions and permit enhanced ventilation and evacuation of perspiration fluids from the eye area.

In view of the aforesaid written description of the present invention, it will be readily understood by those persons skilled in the art that the present invention is susceptible of broad utility and application. Many embodiments and adaptations of the present invention other than those herein described, as well as many variations, modifications, and equivalent arrangements, will be apparent from or reasonably suggested by the present invention and the foregoing description thereof, without departing from the substance or scope of the present invention. Accordingly, while the present invention has been described herein in detail in relation to preferred embodiments, it is to be understood that this disclosure is only illustrative and exemplary of the present invention and is made merely for purposes of providing a full and enabling disclosure of the invention. The foregoing disclosure is not intended nor is to be construed to limit the present invention or otherwise to exclude any such other embodiments, adaptations, variations, modifications and equivalent arrangements, the present invention being limited only by any claims appended hereto and the equivalents thereof.

What is claimed is:

1. A protective eyewear system suitable for use to protect a human user during athletic competitions, the user having eyes, eye sockets, a face, and a nose having a bridge portion, comprising:
a hybrid frame that includes an elastomerically flexible portion and at least one rigid portion having a varying width and being disposed within and adjacent to the flexible portion; and
a pair of lenses each independently supported within one of the at least one rigid portion, the lenses being generally concave to the eyes of the user;
wherein the width of the at least one rigid portion varies between a minimum sufficient to support the lens and one or more maxima denoting an armature, the armatures being disposed to correspond to and interface with selected portions of the eye socket of the user at which pressure may be safely applied in the event of impact of the eyewear with another object; and
wherein at least one rigid portion is configured to be generally convex to the eye and contoured to the face of the user.

2. A system according to claim 1, wherein the flexible portion of the hybrid frame is configured to connect the two lenses to permit them to flex and float each independently of the other.

3. A system according to claim 1, wherein the flexible portion of the hybrid frame is configured to interpose a section of the flexible portion between the rigid portion and the user in order to attenuate shock upon impact with the object.

4. A system according to claim 1, wherein at least one of the armatures comprises at least one ventilation aperture for evacuating fluids from and ventilating the region behind the lens to prevent fogging and accumulation of fluids.
